(19) United States
(12) Patent Application Publication
(10) Pub. No.: US 2018/0214132 A1
(43) Pub. Date: Aug. 2, 2018

(54) METHOD OF ULTRASONIC IMAGING

(71) Applicant: General Electric Company, Schenectady, NY (US)

(72) Inventor: Hanako Kato, Hino (JP)

(21) Appl. No.: 15/885,256

(22) Filed: Jan. 31, 2018

(30) Foreign Application Priority Data
Jan. 31, 2017 (JP) 2017-015254

(51) Int. Cl. A61B 8/00; A61B 8/08

(52) U.S. Cl. CPC A61B 8/469; G06T 2207/10132; A61B 8/461; A61B 8/481

(57) ABSTRACT

A method of ultrasonic imaging includes displaying a first ultrasonic image, receiving an operator's input defining a region of interest, creating a first time intensity curve for the region of interest in the first ultrasonic image, displaying the first time intensity curve on a display device with the first ultrasonic image, and storing information on a position of the region of interest in a non-transitory storage medium. The method includes displaying a second ultrasonic image, reading information on the position from the non-transitory storage medium, creating a second time intensity curve for the region of interest in the second ultrasonic image; and displaying the second time intensity curve on the display device with the second ultrasonic image.

### FIELD OF INVENTION

The present invention relates to a method for displaying a curve of a temporal change of brightness, i.e., a TIC (Time Intensity Curve) in a region of interest defined in an ultrasonic image.

### BACKGROUND OF INVENTION

A TIC is a graph having average signal intensity for a region of interest defined in an ultrasonic image on a vertical axis and time on a horizontal axis. For example, when it is desired to observe a difference in inflow and outflow of a contrast medium between the inside and outside of a tumor, a region of interest is defined in a contrast image in the inside and outside of the tumor. By observing the TIC in this condition, a difference in temporal change may be visually/quantitatively evaluated.

### BRIEF DESCRIPTION OF THE INVENTION

Purposes for which the TIC is used include a study purpose and a presentation purpose. The shape of a region of interest and the position where it should be placed have been determined relying upon various opinions including, for example, the opinion that it is preferable to draw a region of interest freehand so that the region has generally the same shape as the contour of a tumor to contain the whole tumor, and the opinion that it is sufficient to place a circular region of interest small enough in the inside of a tumor. Therefore, elaborate processing operations, such as shaping or positioning of a region of interest according to the shape of a tumor or the like are sometimes required, which may take several days. Moreover, after once completing an operation of defining the region of interest to observe a TIC, the operator may sometimes desire to resume the operation and change the position of the previously defined region of interest or add another region of interest.

As such, the operation of defining or editing a region of interest for which a TIC is to be created may sometimes require resumption after once completing it. In this case, the operation is conventionally redone from the beginning, causing significantly lowered operational efficiency.

In one aspect, the invention made for solving the aforementioned problem is an ultrasonic diagnostic apparatus comprising: a display device for displaying thereon a first ultrasonic image of a subject; an input device for accepting an operator's input for defining a region of interest in said first ultrasonic image; a creating section for creating a first time intensity curve in said region of interest; and a non-transitory storage medium for storing therein information on a position of said region of interest on said display device and data for said first ultrasonic image, wherein said creating section further creates, after reading said information on a position and said data stored in said non-transitory storage medium, a second time intensity curve in said region of interest whose position is located based on said information on a position in a second ultrasonic image displayed on said display device based on said data.

According to the invention in the aspect described above, information on a position of a region of interest defined in a first ultrasonic image and data for the first ultrasonic image are stored in a non-transitory storage medium, and a position of the region of interest in a second ultrasonic image displayed on the display device based on the data is located based on the information on a position. Then, a second time intensity curve in the region of interest whose position is thus located is created. Therefore, in the case that after completing an operation of defining a region of interest once, the operator resumes the operation, the region of interest is defined at the position before completion, and therefore, the need for redoing the operation from the beginning is eliminated, thus improving operational efficiency.

### BRIEF DESCRIPTION OF THE DRAWINGS

FIG. 1 is a block diagram showing a general configuration of an ultrasonic diagnostic apparatus in accordance with the present invention.

FIG. 2 is a block diagram showing a configuration of a display processing section in the ultrasonic diagnostic apparatus shown in FIG. 1.

FIG. 3 is a diagram showing a display device on which an ultrasonic image having a region of interest defined therein is displayed.

FIG. 4 is a block diagram showing a storage device.

FIG. 5 is a flow chart explaining creation of a first time intensity curve.

FIG. 6 is a diagram showing the display device on which the first time intensity curve is displayed.

FIG. 7 is a flow chart explaining creation of a second time intensity curve.

FIG. 8 is a diagram showing the display device on which the second time intensity curve is displayed.

FIG. 9 is a block diagram showing a configuration of the display processing section in a variation.

FIG. 10 is a diagram explaining tracking.

### DETAILED DESCRIPTION OF THE INVENTION

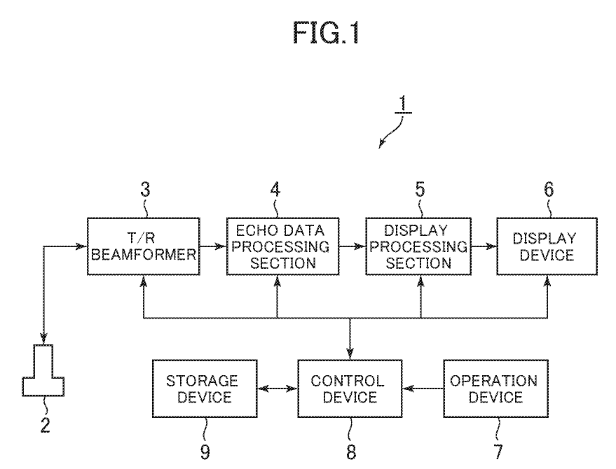

Now an embodiment of the present invention will be described with reference to the drawings. An ultrasonic diagnostic apparatus 1 shown in FIG. 1 comprises an ultrasonic probe 2, a transmission/reception (T/R) beamformer 3, an echo data processing section 4, a display processing section 5, a display device 6, an operating device 7, a control device 8, and a storage device 9. The ultrasonic diagnostic apparatus 1 has a configuration as a computer.

The ultrasonic probe 2 is configured to have a plurality of ultrasonic vibrators (omitted in the drawing) arranged in an array, and it is by the ultrasonic vibrators that ultrasound is transmitted to a subject and echo signals therefrom are received.

The T/R beamformer 3 supplies to the ultrasonic probe 2 an electric signal for transmitting ultrasound from the ultrasonic probe 2 under specific scan conditions, based on a control signal from the control device 8. The T/R beamformer 3 also performs signal processing, such as A/D conversion and phased addition processing, on the echo signals received by the ultrasonic probe 2, and outputs signal-processed echo data to the echo data processing section 4.

The echo data processing section 4 performs processing for producing an ultrasonic image on the echo data output from the T/R beamformer 3. For example, the echo data processing section 4 performs B-mode processing, such as logarithmic compression processing and envelope detection processing, to create B-mode data.

Moreover, the echo data processing section 4 may perform processing for producing a contrast image in which a contrast medium administered to the subject is enhanced, on the echo data output from the T/R beamformer 3 to create contrast data. For example, the echo data processing section 4 performs filtering for extracting harmonic components in echo signals. The echo data processing section 4 may perform processing of extracting echo signals of the contrast medium according to a pulse inversion technique. The echo data processing section 4 may also perform processing (amplitude modulation) for extracting signal components of the contrast medium by transmitting ultrasound of different amplitudes and subtracting between echo data based on resulting echo signals.

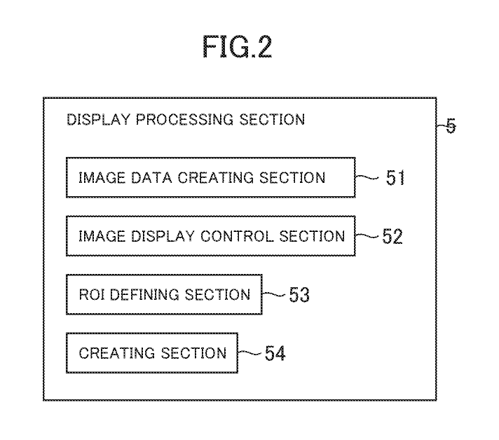

The display processing section 5 comprises an image data creating section 51, an image display control section 52, a region-of-interest (ROI) defining section 53, and a creating section 54, as shown in FIG. 2. The image data creating section 51 scan-converts the data input from the echo data processing section 4 by a scan converter to create ultrasonic image data.

As used herein, the data before being scan-converted into the ultrasonic image data will be referred to as raw data. When the raw data is the B-mode data, B-mode image data is created as the ultrasonic image data. When the raw data is the contrast data, contrast image data is created as the ultrasonic image data.

The image display control section 52 may also create combined image data as the ultrasonic image data, which is obtained by adding the B-mode image data and contrast image data together in a required proportion of addition. The image display control section 52 may create combined image data by creating combined data in which the B-mode data and contrast data are added together in a required proportion of addition, and scan-converting the combined data.

The image display control section 52 displays an ultrasonic image on the display device 6 based on the ultrasonic image data. The ultrasonic image is, for example, a B-mode image based on the B-mode image data or a contrast image based on the contrast image data. The ultrasonic image may also be a combined image based on the combined image data.

The image display control section 52 displays a time intensity curve, i.e., a TIC, for the contrast image on the display device 6. The image display control section 52 is an exemplary embodiment of the image display control section in the present invention. The function by the image display control section 52 is an exemplary embodiment of the image display control function in the present invention.

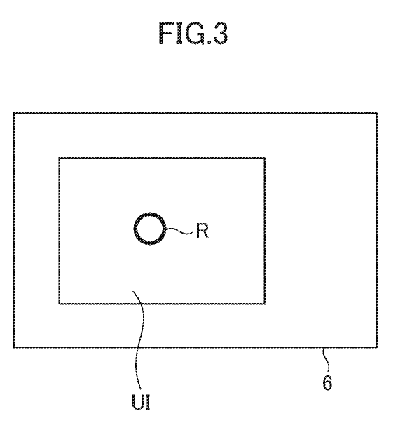

The ROI defining section 53 defines a region of interest R in an ultrasonic image UI displayed on the display device 6, as shown in FIG. 3. The ultrasonic image UI is a combined image, for example. The ROI defining section 53 defines the region of interest R based on, for example, an operator's input at the operating device 7. The function of the ROI defining section 53 is an exemplary embodiment of the region-of-interest defining function in the present invention.

The creating section 54 creates a time intensity curve for the ultrasonic image UI in the region of interest R. Details thereof will be discussed later. The creating section 54 is an exemplary embodiment of the creating section in the present invention. The function of the creating section 54 is an exemplary embodiment of the creating function in the present invention.

The display device 6 is an LCD (Liquid Crystal Display), an organic EL (Electro-Luminescence) display, or the like. The display device 6 is an exemplary embodiment of the display device in the present invention.

The operation device 7 is an input device for accepting an operator's input. For example, the operation device 7 is configured to include buttons, a keyboard, etc. for accepting an input of a command and information from the operator, and to further include a pointing device, such as a trackball, and the like. For example, the operating device 7 accepts an operator's input for defining the region of interest R. The operating device 7 is an exemplary embodiment of the input device in the present invention.

The control device 8 is circuitry for controlling the ultrasonic diagnostic apparatus 1, and is, for example, a processor such as a CPU (Central Processing Unit). The control device 8 loads thereon programs stored in the storage device 9 to control several sections in the ultrasonic diagnostic apparatus 1. For example, the control device 8 loads thereon programs stored in the storage device 9, and executes the functions of the T/R beamformer 3, echo data processing section 4, and display processing section 5 by the loaded programs.

The control device 8 may execute all of the functions of the T/R beamformer 3, all of the functions of the echo data processing section 4, and all of the functions of the display processing section 5 by the programs, or execute only part of the functions by the programs. In the case that the control device 8 executes only part of the functions, the remaining functions may be executed by hardware, such as circuitry, other than the control device 8.

It should be noted that the functions of the T/R beamformer 3, echo data processing section 4, and display processing section 5 may be implemented by hardware, such as circuitry, other than the control device 8.

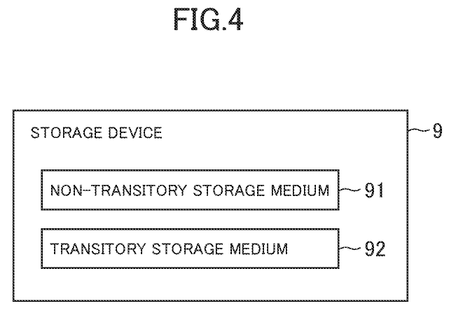

The storage device 9 comprises a non-transitory storage medium 91 and a transitory storage medium 92, as shown in FIG. 4. The non-transitory storage medium 91 is, for example, an HDD (Hard Disk Drive), ROM (Read Only Memory), and the like. The non-transitory storage medium 91 may include a portable storage medium, such as a CD (Compact Disk) or a DVD (Digital Versatile Disk). The programs executed by the control device 8 are stored in the non-transitory storage medium 91. The non-transitory storage medium 91 is an exemplary embodiment of the non-transitory storage medium in the present invention.

The transitory storage medium 92 is RAM (Random Access Memory), and the like.

Now an operation of the ultrasonic diagnostic apparatus 1 in the present embodiment will be described. First, ultrasound transmission/reception is performed by the ultrasonic probe 2 to/from a subject into which a contrast medium is injected, and echo data in a plurality of frames are acquired, based on which raw data in the plurality of frames are created. The raw data is, for example, B-mode data, contrast data, and combined data. The raw data in the plurality of frames are stored in the non-transitory storage medium 91 or transitory storage medium 92. A B-mode image based on the B-mode data and a contrast image based on the contrast data, or a combined image in which the images are combined together may be displayed on the display device 6.

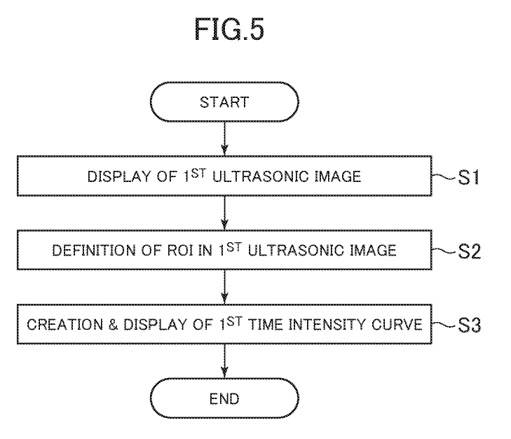

Next, creation of a time intensity curve (TIC) based on the raw data stored in the non-transitory storage medium 91 or transitory storage medium 92 will be described with reference to the flow chart in FIG. 5. The creation of the time intensity curve may be performed in the presence or absence of the subject from which the raw data has been acquired. In the case that the creation is performed in the absence of the subject, the raw data is stored in the non-transitory storage medium 91.

First, at Step S1, the image display control section 52 displays on the display device 6 an ultrasonic image UI based on the raw data stored in the non-transitory storage medium 91 or transitory storage medium 92. For example, the image display control section 52 may display a combined image as the ultrasonic image UI. However, the ultrasonic image UI is not limited to the combined image. The ultrasonic image UI displayed here will be referred to as first ultrasonic image UI1.

Next, at Step S2, the ROI defining section 53 defines a region of interest R in the first ultrasonic image UI1 displayed on the display device 6. Once the operating device 7 has accepted an operator's input for defining the region of interest R, the ROI defining section 53 performs definition of the region of interest R. Moreover, once the operating device 7 has accepted the operator's input for defining the region of interest R, the control section 8, for example, stores in the non-transitory storage medium 91 information on a position of the region of interest R on the display device 7. The information on a position of the region of interest R is stored in connection with the raw data. The function of the aforementioned storage by the control section 8 is an exemplary embodiment of the storing function in the present invention.

It should be noted that the timing of when to store the information on a position of the region of interest R is not limited to the aforementioned timing. For example, the information on a position of the region of interest R may be stored when a time intensity curve C is created at Step S3.

Next, at Step S3, the creating section 54 creates a time intensity curve C in the region of interest R defined at Step S2. The time intensity curve C is a curve representing a temporal change of brightness of the ultrasonic image UI, which is a temporal change of the average brightness, for example, within the region of interest R. More specifically, the creating section 54 creates the time intensity curve C based on values of the ultrasonic image data (combined image data or contrast image data) or raw data (combined data or contrast data).

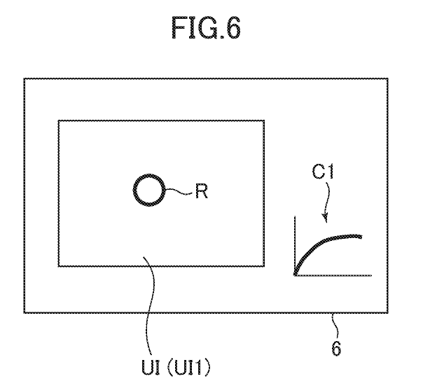

The time intensity curve C here will be referred to as first time intensity curve C1. The image display control section 52 displays the first time intensity curve C1 side by side with the first ultrasonic image UI on the display device 6, as shown in FIG. 6.

In the case that the raw data is stored only in the transitory storage medium 92, the raw data is stored in the non-transitory storage medium 91 at Step S3, for example. The non-transitory storage medium 91 may also store therein the ultrasonic image data.

The non-transitory storage medium 91 may also store therein conditions for creating the first time intensity curve C1. The conditions for creating the first time intensity curve C1 may include, for example, one required to draw the time intensity curve, such as a curve rendering method (a function, etc.).

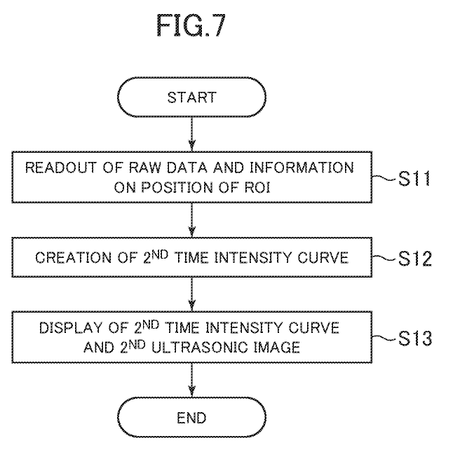

Next, a case in which after the first time intensity curve C1 is displayed at Step S3, the operator completes the operation once and then resumes display of the time intensity curve will be described with reference to the flow chart in FIG. 7. First, at Step S11, the creating section 54 reads the raw data and information on a position of the region of interest R stored in the non-transitory storage medium 91. Next, at Step S12, the creating section 54 creates a time intensity curve C in the region of interest R whose information on a position is read from the non-transitory storage medium 91, based on the raw data read from the non-transitory storage medium 91. The time intensity curve C created here will be referred to as second time intensity curve C2.

The creating section 54 uses the information on a position read from the non-transitory storage medium 91 to identify ultrasonic image data or raw data of a portion for which the second time intensity curve C2 is to be created, and creates the second time intensity curve C2. Moreover, the creating section 54 creates the second time intensity curve C2 under the conditions stored in the non-transitory storage medium 91.

The data for which the second time intensity curve C2 is to be created in the ultrasonic image data or raw data is data of a portion in which the region of interest R is defined in the ultrasonic image displayed at Step S13 discussed later. Therefore, identifying data of a portion for which the second time intensity curve C2 is to be created is equivalent to locating the position of the region of interest in the ultrasonic image.

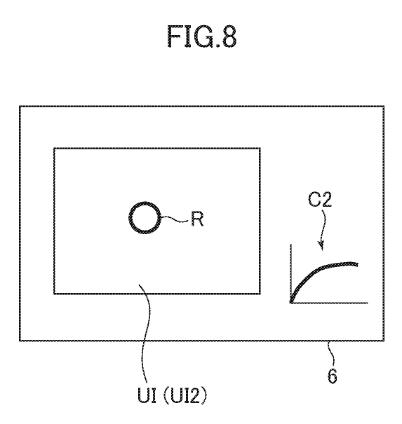

Next, at Step S13, the image display control section 52 displays the second time intensity curve C2 created at Step S12 on the display device 6, as shown in FIG. 8. The image display control section 52 also displays the ultrasonic image UI based on the raw data read at Step S1 side by side with the second time intensity curve C2. The ultrasonic image UI is a combined image, for example. The ultrasonic image UI displayed here will be referred to as second ultrasonic image UI2. The second ultrasonic image UI2 is the same image as the first ultrasonic image UI1. Second ultrasonic images UI2 in a plurality of frames may be displayed.

The ROI defining section 53 displays the region of interest R in a portion located at Step S2 in the second ultrasonic image UI2.

At this time, the operator may perform an input at the operating device 7 for modifying the region of interest R displayed on the display device 6. According to the input, the ROI defining section 53 modifies the region of interest R. The modification of the region of interest R may include modification of the position of the region of interest R or modification of the shape of the region of interest R.

The operator may also perform an input at the operating device 7 for defining a new region of interest in the second ultrasonic image UI2. According to the input, the ROI defining section 53 refreshes definition of the region of interest.

According to the embodiment described above, even in the case in creating a time intensity curve C that the operator cannot complete on a single try a processing operation for obtaining a region of interest at an optimal position and of an optimal shape or an operation of defining a region of interest for which a time intensity curve C suitable for diagnosis is to be obtained, and resumes the operation after completing it, he/she does not have to redo the operation from the beginning. Thus, operational efficiency may be improved. Moreover, the operator can freely suspend operations.

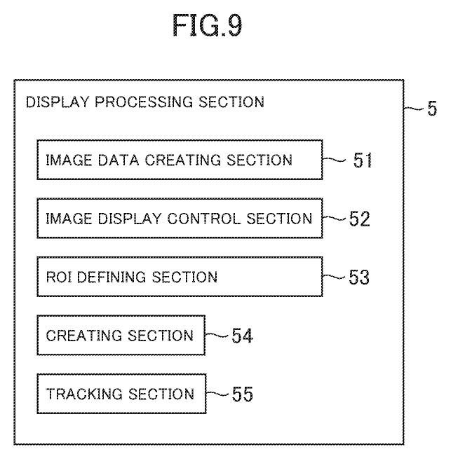

Next, a variation of the embodiment will be described. As shown in FIG. 9, the display processing section 5 may comprise a tracking section 55. The tracking section 55 tracks the portion in which the region of interest R is defined in each of the first ultrasonic images UI1 and second ultrasonic images UI2 across a plurality of frames. In the case that the same portion in the subject moves in ultrasonic images UI across a plurality of frames caused by the subject's body motion or movement of the ultrasonic probe 2, the tracking section 55 tracks the movement to follow the region of interest. For example, the tracking section 55 calculates a correlation between B-mode images in two frames to detect movement of the portion in which the region of interest R is defined. The tracking section 55 is an exemplary embodiment of the tracking section in the present invention.

In this variation, once the region of interest R has been defined in the first ultrasonic image UI1 at Step S2, frame information for identifying a frame of the first ultrasonic image UI1 for which the operator has defined the region of interest R is stored in the non-transitory storage medium 91.

Moreover, at Step S3, the tracking section 55 tracks the portion in which the region of interest R is defined in each of the first ultrasonic images UI1 across a plurality of frames. The tracking section 55 may perform tracking in the B-mode image data from which the first ultrasonic image UI1 is produced. The creating section 54 then creates the first time intensity curve C1 in the region of interest R whose position is located by tracking by the tracking section 55.

The non-transitory storage medium 91 stores therein information indicating whether or not the tracking has been performed by the tracking section 55 at Step S3. The operator may perform an input at the operating device 7 for selecting whether to enable or disable the tracking function by the tracking section 55.

Moreover, in this variation, at Step S12, when information indicating that the tracking has been performed at Step S3 is stored, the tracking section 55 tracks the portion in which the region of interest R is defined in each of the second ultrasonic images UI2 across a plurality of frames. The tracking section 55 may perform tracking in the B-mode image data from which the second ultrasonic image UI2 is produced.

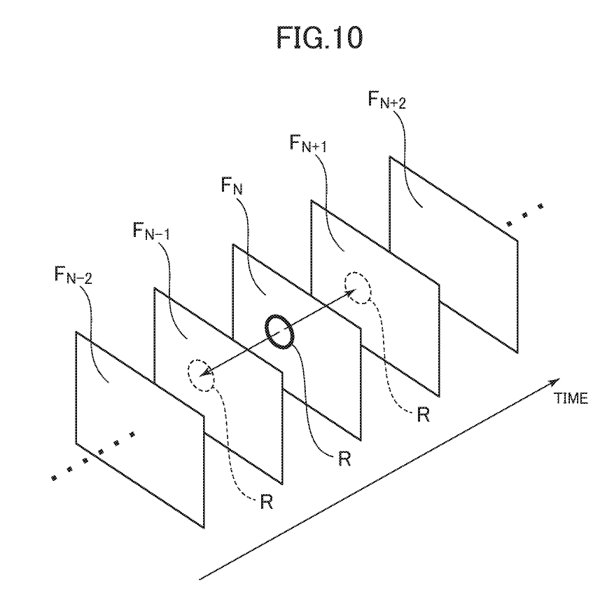

The tracking section 55 starts tracking with reference to the position of the region of interest R in the second ultrasonic image UI2 in the same frame as that identified by the frame information stored in the non-transitory storage medium 91. For example, in the case that the frame information stored in the non-transitory storage medium 91 indicates an N^{th} frame, the tracking section 55 starts tracking with reference to the position of the region of interest R in the second ultrasonic image UI2 in the N^{th} frame F_{N}, as shown in FIG. 10. Specifically, the tracking section 55 calculates a correlation between the frame F_{N} and its immediately preceding frame F_{N-1} to calculate movement of the region of interest R between the frame F_{N} and frame F_{N-1}. The tracking section 55 also calculates a correlation between the frame F_{N} and its immediately following frame F_{N+1} to calculate movement of the region of interest R between the frame F_{N} and frame F_{N-1}.

Once the position of the region of interest R has been located in the frame F_{N-1}, the tracking section 55 calculates movement of the region of interest R between the frame_{N-1} and frame_{N-2}. Similarly thereafter, the tracking section 55 calculates movement of the region of interest R up to a first frame (a frame acquired temporally first) to locate its position. Moreover, once the position of the region of interest R has been located in the frame F_{N+1}, the tracking section 55 calculates movement of the region of interest R between the frame_{N+1} and frame_{N+2}. Similarly thereafter, the tracking section 55 calculates movement of the region of interest R down to a last frame (a frame acquired temporally last) to locate its position.

The creating section 54 creates, at Step S12, a second time intensity curve C2 in the region of interest R whose position is located by tracking by the tracking section 55. At Step S13, the second time intensity curve C2 in the tracked region of interest R is displayed.

Moreover, in the case that second ultrasonic images UI2 in a plurality of frames are displayed at Step S13, the ROI defining section 53 displays the region of interest R whose position is located by tracking by the tracking section 55 in each of the second ultrasonic images UI2 in the plurality of frames.

According to this variation, even in the case that the subject's body motion or movement of the ultrasonic probe 2 is encountered, a first time intensity curve C1 before suspending operations and a second time intensity curve C2 after the suspension may be displayed as time intensity curves in the same portion in the subject.

While the present invention has been described with reference to the embodiments, it will be easily recognized that the present invention may be practiced with several modifications without changing the spirit and scope thereof. For example, on the display device 6, a B-mode image and a contrast image may be displayed side by side. In this case, for example, the operator may define a region of interest in the B-mode image; moreover, a time intensity curve at a position in the contrast image corresponding to the position of the region of interest may be created.

We claim:

1. A method of ultrasound imaging comprising:
displaying a first ultrasonic image based on a subject on a display device, where the first ultrasonic image is based on ultrasonic data;
receiving an operator's input through an input device for defining a region of interest in the first ultrasonic image;
creating a first time intensity curve for the first ultrasonic image in the region of interest;
displaying the first time intensity curve on the display device with the first ultrasonic image;
storing information on a position of the region of interest in a non-transitory storage medium;
displaying a second ultrasonic image of the subject on the display device, where the second ultrasonic image is based on the ultrasonic data;
reading the information on the position from the non-transitory storage medium;
creating a second time intensity curve for the second ultrasonic image in the region of interest, where the position of the region of interest is based on the information read from the non-transitory storage medium; and
displaying the second time intensity curve on the display device with the second ultrasonic image.

2. The method of claim 1, further comprising displaying an image display control section on the display device.

3. The method of claim 1, further comprising receiving an input to modify the region of interest in the second ultrasonic image.

4. The method of claim 1, further comprising receive an input for defining a new region of interest in the second ultrasound image.

5. The method of claim 1, further comprising simultaneously displaying both the first time intensity curve and the second time intensity curve on the display device.

6. The method of claim 1, wherein the ultrasonic data comprises a plurality of frames and further comprising tracking the position of the region of interest in the plurality of frames.

7. The method of claim 1, where the ultrasonic data comprises raw data.

8. The method of claim 1, where the ultrasonic data is obtained by scan-converting the raw data.

9. The method of claim 1, further comprising storing conditions for creating the first time intensity curve in the non-transitory storage medium.

10. The method of claim 9, further comprising reading the conditions for creating the first time intensity curve from the non-transitory storage medium, and wherein said creating the second time intensity curve comprises creating the second time intensity curve under the conditions for creating the first time intensity curve stored in the non-transitory storage medium.
